(19) United States
(12) Patent Application Publication
(10) Pub. No.: US 2004/0146564 A1
(43) Pub. Date: Jul. 29, 2004

(54) Process for making delivery matrix and uses thereof

(76) Inventors: Muriel Subirade, Sillery (CA); Lucie Beaulieu, Laval (CA); Paul Paquin, Charny (CA)

Correspondence Address: OGILVY RENAULT, 1981 MCGILL COLLEGE AVENUE, SUITE 1600, MONTREAL, QC, H3A2Y3, CA

(21) Appl. No.: 10/471,603

(22) PCT Filed: Apr. 5, 2002
(86) PCT No.: PCT/CA02/00461

(51) Int. Cl. A61K 9/50; B29C 39/10

(52) U.S. Cl. 424/499; 264/4

(57) ABSTRACT

The present invention relates to a process of making particles for cutaneous application or oral delivery of bioactive molecules or living systems. The process can be carried out in some simple steps. The present invention further discloses a new physiological composition allowing release different nutraceutical, biological, cosmetic, cosmeceutical, or therapeutical compounds in a human, an animal, or a composition.

### BACKGROUND OF THE INVENTION

(a) Field of the Invention

The invention relates to processes for producing particles of composed proteins. The present invention also pertains to a new oral delivery system incorporating biologically active material and to preparations of such system containing biologically useful compounds, particularly hydrophobe molecules, nutraceutical and therapeutical agents.

(b) Description of Prior Art

Particles and microcapsules have important applications in the pharmaceutical, agricultural, textile and cosmetics industry as delivery vehicles. In these fields of application, many compounds such as drugs, proteins, hormones, peptides, fertilizers, pesticides herbicides, dyes, fragrances or other agents can be encapsulated in a polymer matrix to be delivered in a site either instantaneously or in a controlled manner in response to some external impetus (i.e., pH, heat, water, radiation, pressure, concentration gradients, etc.).

Many encapsulation techniques exist to produce a variety of sphere types and sizes under various conditions. Methods typically involved for solidifying emulsified liquid polymer droplets by changing temperature, evaporating solvent, or adding chemical cross-linking agents. Physical and chemical properties of the encapsulant and the material to be encapsulated can sometimes dictate the suitable methods for encapsulation, making only certain methodologies useful in some circumstances. Factors such as hydrophobicity, molecular weight, chemical stability, and thermal stability affect encapsulation. Significant losses are frequently associated with several processing steps. These parameters can be particularly important concerning encapsulation of bioactive molecules because losses in the bioactivity of the material due to the processing steps or low yields can be extremely undesirable.

Common encapsulation techniques include interfacial polycondensation, spray drying, hot melt encapsulation, and phase separation techniques (solvent removal and solvent evaporation). Interfacial polycondensation can be used to microencapsulate a core material in the following manner. One monomer and the core material are dissolved in a solvent. A second monomer is dissolved in a second solvent (typically aqueous) which is immiscible with the first. Suspending the first solution through stirring in the second solution forms an emulsion. Once the emulsion stabilized, an initiator is added to the aqueous phase causing interfacial polymerization at the interface of each droplet of emulsion.

The increasing interest in effective and selective delivery of bioactive molecules into the site of action has led to the development of new encapsulation materials. Despite the successful elaboration of many synthetic polymers as biodegradable microencapsulating media, natural polymers remain attractive agents that are extensively investigated. These materials have the potential advantages of great availability, low cost, low toxicity, and the ability to be easily modified. Although many wall materials are available for non-food applications, very few are used in food applications. However, among the systems investigated, food proteins have recently received considerable attention because of their excellent functional properties. Proteins, such as gelatin, gliadin, human serum albumin, or egg albumin, have been used with success for encapsulating bioactive molecules.

Whey proteins, also known as the serum proteins of milk, are widely used in food products because of their high nutritional value and their ability to form gels, emulsions, or foams. It is known that, using a spray-drying technique, that whey proteins form spherical microcapsules. However, this technique involves high temperatures during the drying process and, consequently, limits its use to active, heat-resistant materials. Another methods which is based on an emulsification with glutaraldehyde cross-linking, has been developed for using whey protein particles. However, it has the disadvantages of requiring the use of an organic solvent, of being difficult to remove from the finished product, and of using glutaraldehyde, which restricts it out of the biomedical field because of its toxic effects.

The U.S. Pat. Nos. 5,091,187 and 5,091,188 describe the use of phospholipids as surface stabilizers to produce aqueous suspension of submicron sized particles of the water-insoluble drugs. These suspensions are believed to be the first applications of the surface modified microparticulate aqueous suspension containing particles made up with a core of pure drug substances and stabilized with natural or synthetic bipolar lipids including phospholipids and cholesterol.

Subsequently, similar delivery systems exploiting these principles have been described (G. G. Liversidge et al., U.S. Pat. No. 5,145,684; K. J. Illig et al. U.S. Pat. No. 5,340,564 and H. William Bosch et al., U.S. Pat. No. 5,510,118) emphasizing the usefulness of the drug delivery approach utilizing particulate aqueous suspensions.

The U.S. Pat. No. 5,246,707 demonstrates the uses of phospholipid-coated microcrystals in the delivery of water-soluble biomolecules such as polypeptides and proteins. The proteins are made insoluble by complexation and the resulting material forms the solid core of the phospholipid-coated sphere.

Among the alternatives that address these problems there is a procedure that uses liquefied gasses for the production of microparticulate preparations. In such a method, liquefied-gas solutions are sprayed to form aerosols from which fine solid particles precipitate.

It would be highly desirable to be provided with a new method for producing particles able to act as delivery system of bioactive molecules or systems into different organisms or compositions.

### SUMMARY OF THE INVENTION

One object of the present invention is to provide a new method for producing particles that can be used as delivery systems of physiologically active molecules, into an organism, such as but not limited to animals, and humans.

Another object of the present invention is to provide particles for delivery of bioactive molecules and systems, bacteria, mycorhizes, mould, and other microorganisms as pre- and probiotics.

In accordance with the present invention a process for making particles for delivery of a bioactive molecule or system is provided comprising the steps of:

a) providing a solution of protein;

b) heating the solution of step a) to a temperature sufficient to allow denaturation of the protein, the heating occurring at a temperature of about 20° C. to 150° C. for a period of at least 2 minutes to 10 hours;

c) adding an hydrophobic phase to the heated solution of step b) in a ratio of about 5 to 60 percents (vol/vol) to form a mixture so that an emulsion is formed;

d) homogenizing the emulsion of step c); and

e) contacting the homogenized emulsion of step d) with a salt solution so that particles are formed.

The proteins may be selected from the group consisting of synthetic peptide, milk protein, whey protein, vegetable protein, bran protein, animal protein, and globular peptide or protein.

The heated solution may further be cooled down before the addition of a hydrophobic phase.

The homogenization of the process may be performed under dynamic high pressure or mechanical homogenization.

At least one physiological agent, bioactive molecule, or system may be added to the particles during the preparation process.

The system may be selected from the group consisting of bacteria, virus, mould, yeast, semen, pollen, grain, and microorganism.

The hydrophobic phase may be selected from the group consisting of oil, physiologically acceptable carrier, adjuvant, emulsifier, diluent or excipient.

The oil may be selected from the group consisting of animal, mineral, and vegetable oil.

The bioactive molecule may be selected from the group consisting of nutraceutical, immunological, enzymatic, cosmetic, cosmeceutical, and therapeutical agents.

The bioactive molecule may be selected from the group consisting of nutritional products, mucopolysaccharides, vitamins, anti-oxidants, lipids, laxatives, carbohydrates, steroids, hormones, growth hormone (GH), growth hormone releasing hormone (GHRH), epithelial growth factor, vascular endothelial growth and permeability factor (VEGPF), nerve growth factor, cytokines, interleukins, interferons, GMCSF, hormone-like product, neurological factor, neurotropic factor, neurotransmitter, neuromodulator, enzyme, antibody, peptide, protein fragment, vaccine, adjuvant, an antigen, immune stimulating or inhibiting factor, heomatopoietic factor, anti-cancer product, anti-inflammatory agent, anti-parasitic compound, anti-microbial agent, nucleic acid fragment, plasmid DNA vector, cell proliferation inhibitor or activator, cell differentiating factor, blood coagulation factor, immunoglobulin, a histamine receptor antagonist anti-angiogenic product, negative selective markers or “suicide” agent, toxic compound, anti-angiogenic agent, polypeptide, anti-cancer agent, acid production drug, probiotic, prebiotic, a microorganism, a mould, a yeast, a mycorhize, a rhizobacteria.

The delivery of bioactive molecules or systems may be carried out under form of cutaneous application or oral administration.

The delivery may also be performed in a subject or a composition, wherein the subject is a human or an animal, and the composition may be an organic mixture, a fertilizer, manure, an earth, a ground, or a land.

The salt that may be used to perform the process of the present invention may be a soluble salt selected from the group consisting of divalent cations, calcium chloride, sodium chloride, calcium phosphate, sodium phosphate, sodium carbonate, potassium carbonate, calcium sulfate, carboxylic acid, salts, barium, magnesium, calcium, iron, and derivatives thereof.

In accordance with the present invention a particle as obtained with the method for delivery of a bioactive molecule or system to a subject or a composition is provided.

In accordance with the present invention a method for delivery of a bioactive molecule or a system to a subject or a composition comprising delivery to a subject or a composition particles as obtained by the method of the present invention, and containing bioactive molecules or systems is also provided.

The delivery may occur under the form of cutaneous application, oral administration, or mixing fertilizer, earth, land or ground.

For the purpose of the present invention the following terms are defined below.

As used herein, the term “protein” is intended to refer to compounds composed, at least in part, of amino acid residues linked by amide bonds (i.e., peptide bonds). The term “protein” is intended to include peptides, and polypeptides. The term “protein” is further intended to include peptide analogues, peptide derivatives and peptidomimetics that mimic the chemical structure of a protein composed of naturally occurring amino acids. Examples of peptide analogues include peptides comprising one or more non-natural amino acids. Examples of peptide derivatives include peptides in which an amino acid side chain, the peptide backbone, or the amino- or carboxy-terminus has been derived (e.g., peptidic compounds with methylated amide linkages). The terms “protein”, “peptide” and “polypeptide” refer to both naturally occurring chemical entities and structurally similar bioactive equivalents derived from either endogenous, exogenous, or synthetic sources and is used to mean polymers of amino acids linked together by an amide type linkage known as a peptide bond.

As used herein, the term “bioactive molecule” is intended to refer to a peptide or a molecule that exhibits biological, biochemical, nutraceutical, or pharmacological activity, either in its present form or upon processing in vivo (i.e., pharmaceutically active peptidic compounds include peptidic compounds with constitutive pharmacological activity and peptidic compounds in a “prodrug” form that have to be metabolized or processed in some way in vivo following administration in order to exhibit pharmacological activity). The term bioactive molecule is intended to include also vitamins, peptides, prebiotics, and probiotics.

The term “system” as used herein refers to living systems capable of inducing a biological, biochemical, or chemical reaction into a host animal or human. It includes, without limitation, bacteria, mould, yeast, viruse, and any other microorganism. The system may be considered as a probiotic or prebiotic system.

The term “therapeutic agent” is used in a generic sense and includes treating agents, prophylactic agents, replacement agents, and antimicrobial agents.

The term “mucosal immune system” refers to the fact that immunization at any mucosal site can elicit an immune response at all other mucosal sites.

The term “particle” or “sphere” as used throughout the specification includes particles and microcapsules and refers to a small particle ranging in size from 5 micrometers to 8 millimeters in diameter.

The term “hydrophobic phase” as used herein refers to agents, or products that are insolubles in water, or in solutions principally composed of water. The hydrophobic phases may include, but is not limited to, any oil originating from animal, vegetable or being synthetically obtained, or other products having low water compatibility.

### BRIEF DESCRIPTION OF THE DRAWINGS

FIGS. 1a to 1c show macrophotographs of whey protein beads prepared with 10% CaCl₂ concentration (w/w) (FIG. 1a); prepared with 15% CaCl₂ concentration (FIG. 1b); prepared with 20% CaCl₂ concentration (FIG. 1c);

FIGS. 2a to 2c show a representative TEM image of internal structure of whey protein beads: prepared with 10% CaCl₂ concentration (w/w) (FIG. 2a); prepared with 15% CaCl₂ concentration (FIG. 2b); prepared with 20% CaCl₂ concentration (FIG. 2c);

FIG. 3 shows the swelling ratio (%) of beads as a function of CaCl₂ concentration (10, 15, 20% w/w) and pH (1.9, 4.5, and 7.5);

FIG. 4 illustrates the fracture stress (Nm⁻²) of beads as a function of CaCl₂ concentration (10, 15, 20% w/w) and pH (1.9, 4.5, and 7.5);

FIG. 5 illustrates the fracture strain of beads as a function of CaCl₂ concentration (10, 15, 20% w/w) and pH (1.9, 4.5, and 7.5);

FIG. 6 shows the stress relaxation (%) of beads as a function of CaCl₂ concentration (10, 15, 20% w/w) and pH (1.9, 4.5, and 7.5); and

FIGS. 7a to 7c show macrophotographs of beads: prepared with 20% CaCl₂ concentration (w/w) (FIG. 7a); after a 30-minute gastric incubation (FIG. 7b); after a 6-hour pancreatic incubation (FIG. 7c).

### DETAILED DESCRIPTION OF THE INVENTION

In accordance with the present invention, and to overcome the limitations of existing methods (i.e. high temperature, organic solvents, and toxic agents), a new encapsulation method for encapsulating physiologically active agents which uses proteins there is provided. First, a two-phase process involving an emulsifying step followed by a Ca²⁺-induced gelation of pre-denatured whey protein is described. Beads are then formed by the dropwise addition of suspension into a calcium chloride solution according to the method used to produce calcium-alginate beads. Secondly, the physicochemical and mechanical characterizations of the beads are studied with respect to CaCl₂ concentrations (10, 15, 20% w/w) and pH levels (1.9, 4.5, and 7.5). The swelling ratio, one of the most important factors affecting the drug release characteristics in drug delivery systems, is determined. Indeed, the drug release is dependent on the swelling of the matrix. Thus, the matrix has the ability to release drugs in response to changes in environmental variables such as temperature, pH, ionic strength, etc. As for pH sensitive drug delivery systems, many studies that addressed the relationships between the swelling ratio of the vehicle and the drug release characteristics are reported. Mechanical properties of the beads were also determined since they are of great importance when they have to be used in a bioreactor, implanted in vivo, or used in food processes that possibly undergo different treatments such as cutting, slicing, spreading, or mixing. In the last part of the work, stability assays are carried out with a selected batch of beads using an in vitro protease degradation. Bead susceptibility to some proteolytic enzymes is studied using a two-step proteolysis, which first consisted in the predigesting of beads with pepsin followed by pancreatin.

In one embodiment of the present invention, the protein source used to form matrices with the present method is milk, whey, globular proteins, soybean proteins, and globular proteins.

In one embodiment of the present invention, the particle preparation method does not adversely affect the biological activity of the molecules introduced therein. Therefore, the molecules released from the particles retain their natural bioactivity.

The particles have generally uniform sizes and shapes. The characteristics of the particles may be altered during preparation by manipulating the protein concentration, reaction temperature, pH, and molecule concentration.

In another embodiment of the present invention, particles that are useful for a wide variety of separation, diagnostic, therapeutic, industrial, commercial, cosmetic, and research purposes or for any purpose requiring the incorporation of and stabilization of an active molecule, bioactive molecule, system, reactant, drug, and recombinant or derivative thereof are provided.

Another important functional property of whey proteins is their ability to produce heat-induced gel matrices, capable of holding large amounts of water. Depending on the preparation techniques, gels can exhibit different microstructural properties, which are strongly related to the intimate structure of the aggregates. It is shown that cold-induced gelation of whey proteins can be achieved by adding Ca²⁺ ions to a preheated protein suspension. This method requires a heating step during which the denaturation and polymerization of whey proteins into soluble aggregates occur. A cooling step and a subsequent salt addition, which results in a network formation via Ca²⁺-mediated interactions of soluble aggregates, follows this. Ca²⁺-induced whey proteins cold gelation may be compared to alginate gelation resulting from a dimeric association of glucuronic acid regions with Ca²⁺ in the “egg box” formation. Similarly, a gelation mechanism of cross-linking carboxylate groups with Ca²⁺has been suggested for gelation at ambient temperature of pre-denatured whey proteins.

One embodiment of the present invention is to provide a process for making particles that is relatively simple, rapid, and inexpensive.

Another advantage of the invention is the ability to produce particles characterized by a homogenous size distribution. Such particles will have well defined predictable properties.

Another desired form of the complex particle-bioactive molecule of the first embodiment of the present invention is a particle or microcapsule coupled to a carrier molecule, the particle or microcapsule enclosing a hormone, drug, immunogen, or DNA or RNA (such as ribozyme) component, molecule or analogues thereof.

In another embodiment of the present invention a process for making particles that permits modulation of the kinetic release of molecules introduced therein before administration into an organisms is provided.

In one embodiment, the particles of the invention may be synthesized with the addition of an emulsifier, or an excipient.

In one embodiment of the present invention, a particle that contains a bioactive molecule or a system in admixture with non-toxic pharmaceutically acceptable carriers, which are suitable for the manufacture of drug compositions is provided. These carriers may be for example, inert diluents, such as calcium carbonate, calcium chloride, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

In another embodiment of the present invention particles that may exhibit sustained release of different bioactive molecules or systems is provided.

The particles of the invention can contain pharmaceutically acceptable flavors and/or colors in order to make them more appealing. A composition may contain the particles in form of gel, lotion, ointment, cream and the like and may typically contain a sufficient amount of thickening agent so that the viscosity is from 2500 to 6500 cps, although more viscous compositions, even up to 10,000 cps may be employed.

In one embodiment of the invention, depending on the circumstances, liquid for oral administration may also be prepared. Additionally, liquid compositions are somewhat more convenient to administer, especially to animals, children, particularly small children, and anybody who may have some difficulty swallowing a pill, tablet, capsule or the like, or in a multi-dose situation. Viscous compositions on the other hand can be formulated within the appropriate viscosity range to provide longer contact periods with mucosa, such as the lining of the stomach or intestine than a liquid preparation for oral administration.

Also, the particles of the present invention may be mixed with nontoxic pharmaceutically acceptable carriers, and especially oral carriers. Obviously, the choice of suitable carriers will depend on the exact nature of the particular dosage form, e.g., liquid dosage form, e.g., whether the composition is to be formulated into a solution, a suspension, gel or another liquid form, or solid dosage form (e.g., whether the composition is to be formulated into a pill, tablet, capsule, caplet, time release form or liquid-filled form). The choice of suitable carriers will be apparent to scientists.

The present invention provides particles that can release molecules and systems that have retained their biological and/or biochemical activity.

In addition, the present invention provides particles for use in medical and diagnostic applications, such as drug delivery, vaccination, gene therapy and histopathological or in vivo tissue or tumor imaging.

The preparation process of the invention may include insoluble compounds.

By insoluble or poorly soluble compounds, it is included biologically useful compounds, nutraceutical molecules, pharmaceutically useful compounds and in particular drugs for human and veterinary medicine. Usually, water insoluble compounds are those having a poor solubility in water, that is less than 5 mg/mL at a physiological pH of 6.5 to 7.4.

Examples of some preferred water-insoluble molecules include solid form of molecules, immunosuppressive and immunoactive agents, antiviral and antifungal agents, antineoplastic agents, analgesic and anti-inflammatory agents, antibiotics, anti-epileptics, anesthetics, hypnotics, sedatives, antipsychotic agents, neuroleptic agents, antidepressants, anxiolytics, anticonvulsant agents, antagonists, neuron blocking agents, anticholinergic and cholinomimetic agents, antimuscarinic and muscarinic agents, antiadrenergic and antiarrhythmics, antihypertensive agents, antineoplastic agents, hormones, and nutrients.

Another embodiment of this invention is to provide a method for treating or for preventing a disease, or for modulating physiological parameters in a mammal by administering a nutraceutical or pharmaceutical composition through an intestinal mucous membrane.

In one embodiment, the nutraceutic or therapeutic agent may be a peptide or a protein. In another embodiment, the nutraceutic or therapeutic agent in the composition is infused by oral administration, or cutaneous application.

The present invention will be more easily understood by referring to the following examples, which are rather given to illustrate the invention than to limit its scope.

### EXAMPLE I

### Elaboration and Characterization of Whey Protein Beads for the Encapsulation of Bioactive Molecules

Material and Methods

Materials

Whey protein isolates (WPI) were obtained from Davisco Food International, Inc, (Le Sueur, Minn.). Protein content of WPI was 92.96% (dry matter basis), as determined by the Kjeldahl method (nitrogen X 6.38). Soybean oil used to form the emulsions was purchased from a local commercial store (Metro Co., Canada). The enzymes used in the study were pepsin 1:60,000 from porcine stomach mucosa, crystallized and lyophilized, (Sigma Chemical Company St-Louis, Mo., USA) and pancreatin 5× from hog pancreas (ICN Nutritional Biochemicals Cleveland, Ohio, USA). Thimerosal™ (J. T. Baker, Phillipsburg, N.J., USA) was used to prevent bacterial growth and taurocholic acid, the sodium salt form, (Sigma Chemical Company St-Louis, Mo., USA) was used as an emulsifying agent.

Whey Protein Beads Manufacturing Methods

WPI solution (8%, w/w) was adjusted at pH 8. The solution was heated at 80° C. for 30 minutes and simultaneously mixed at 300 rpm in a cooker (Stephan U. Sohne Gmbh & Co., Germany). After cooling for 1 hour at room temperature (˜between 10° C. to 35° C., the ideal cooling temperature being room temperature, 20 to 24° C.), the solution was stored overnight at 4° C. The following day, the solution was equilibrated to room temperature (˜23° C.) and used to produce the emulsion. Protein concentration and oil proportion in the emulsion were 5.6% and 30%, respectively. Prior to preparing the emulsion, the WPI solution and soybean oil were pre-homogenized and mixed using an Ultra-Turrax™ (Janke & Kunkel, IKA-Labortechnik, Germany). The mixture was then homogenized using an Emulsiflex™-C5 high-pressure homogenizer (AVESTIN Inc., Ottawa, Canada). Emulsion preparation was initially performed at 100 MPa pressure and then at 3 MPa. The resulting emulsion was added dropwise into 100 ml of 10, 15 or 20% (w/w) CaCl₂ solutions, using a hydraulic pump (Allo Kramer Shear Press, model SP 12, Rockville, Md., USA) equipped with a syringe and needle (Terumo Medical Corporation, Elkton, Md., USA). Magnetic stirring was maintained during the gelation. The resulting beads were rinsed with distilled water and dried in P₂O₅.

Bead Morphology Analyses

Observations of external bead structure were taken by macrophotographs using a Minolta™ camera (35-mm XG-M) with a 55-mm macro lens.

Internal Bead Structure Analyses by Transmission Electron Microscopy (TEM)

Beads were fixed with formaldehyde 4% (cacodylate buffer 0.1 M) for 2 hours, dehydrated in graded series of ethanol, embedded in LRWhite™ resin and polymerized under UV. Materials were collected on formvar-coated nickel grids and stained with uranyl acetate and lead citrate. Observations were carried out under a JEOL 1200X electron microscope.

Swelling Experiment or Water Uptake Ability

Predetermined amounts of dried whey protein beads were placed in a monosodium phosphate buffer solution (0.02 M contained NaCl 0.13 M) at different pH values: pH 1.9, which corresponds to acid stomach pH; pH 4.5, which is near the pi of whey protein; and pH 7.5, which represents the physiological intestinal pH. Temperature was maintained at 37° C. in an incubator. After 6 hours, the beads in their equilibrium-swollen state were weighed. The swelling ratios of the beads were determined from the weight change before and after swelling, expressed in percentages:

Swelling ratio or Water uptake ability (%)=[(W_{w}−W_{d})/W_{d}]×100

where W_{w} and W_{d} represent the weight of wet and dry beads, respectively.

Compression Studies

The beads were studied by means of a texture analyzer TA-XT2 version 5.15 (50 N maximum force, precision of 0.001 N; Stable Micro Systems (Haslemere, Surrey, United Kingdom). The apparatus was equipped with a 20-mm diameter cylindrical piston. Each measurement was carried out at room temperature on one bead, which was placed under the piston on a fixed bottom plate. For each CaCl₂ concentration (10, 15, and 20% (w/w)), the measurements were repeated on 2 batches of beads, and on 10 beads per batch.

Rupture Study: Stress and Strain at Fracture

The piston went down, keeping contact with the top of the bead, and flattened the bead at a constant rate of 0.2 mm/s, until it reached 90% of its original height. The force exerted by the bead as a function of displacement was recorded. The return speed of the piston to its initial position after compression was 10 mm/s. The force needed for deformation was recorded as a function of time until fracturing of the bead. A force-compression curve was obtained for each sample and stored in a file for calculation of the fracture properties using the “XT.RAD™ Dimension” software, version 3.7H from Stable micro System.

From each measurement, the stress and strain at fracture were determined. The fracture, stress is associated with the first peak on the graphs representing the force as a function of displacement. Stresses (σ; Nm⁻²) were calculated by dividing the force registered at every point by the corresponding bearing area. For gel beads, the stresses were calculated considering the contact area as the area of a sphere and assuming a dissipation of the internal beads force in all directions. The fracture strain (ε), expresses bead deformability and is calculated as follows:

Fracture strain (ε)=|In(h₀−Δh)/h₀|

where h₀ is the initial height and Δh the change in height. The strain is obtained by relating any strain increase (in an already strained sample) to changes in sample dimension.

Stress Relaxation

The piston went down at the rate of 0.2 mm/s until it reached 50% of deformation at bead rupture. The piston then stayed motionless at this position for 30 seconds, and finally returned to its initial position. From the graphs representing the force versus time, the instantaneous resistance strength (F₁), which is the force measured when the piston had just reached its maximum displacement, and (F₂), the force opposed by the bead after 30 seconds, are obtained. From these values, the elasticity of the sample was calculated as the ratio of F₂ to F₁, expressed as a percentage:

Stress relaxation (%)=(F₁−F₂)/F₁×100

when the value of stress relaxation is high, the elasticity is low and vice versa.

In Vitro Degradation Assays

The enzymatic degradation assay was conducted using a modified version of the method of Gauthier et al. (J. Food Sciences, 1986, 51:960-964). Beads (125 mg protein) were suspended in 15 ml of 0.1N HCl (50 mg/ml Thimerosal™) in a flat-bottom glass tube and stirred magnetically for 10 min at 37° C. The volume of the digestion mixture was adjusted to 20 ml and 0.5 ml of pepsin solution (1 mg/ml 0.1N HCl) was added to start the hydrolysis reaction. The digestion was carried out for 30 min and stopped by raising the pH to 7.5 with NaOH. A concentrated monosodium phosphate solution (1 ml; 0.5 M, contained NaCl 3.25 M, pH 7.5) and taurocholic acid (0.5 ml; 0.25 M) were added and the reaction mixture was adjusted to 25.5 ml with distilled water. The reaction was initiated by adding 0.5 ml of pancreatic enzymes (10 mg/ml) prepared in monosodium phosphate buffer (0.02 M, contained NaCl 0.13 M, pH 7.5). The final volume is 25 ml because the magnetic bar takes up a volume of 1 ml. The digestion was carried out for 6 hours and stopped by placing the tube on ice. The end of lysis was defined as the time it took for all particles to disappear.

Statistical Analysis

The combined effects of CaCl₂ concentration (10, 15, 20%) and pH (1.9, 4.5, 7.5) on swelling, fracture stress, strain and stress relaxation were studied using a factorial experimental design (3×3). Data were analyzed by the Statistical Analysis System (SAS Institute, Inc. Cary, N.C., USA) using the General Linear Model (GLM) procedure for regression analyses, ANOVA procedure for analysis of variance, and the Levine test to verify variance homogeneity. Analysis of variance was used to determine whether the factors and their interaction had a significant effect on the measured properties. Statistical analyses were performed at an a=0.05. Error bars on graphs represent standard error obtained from the statistical model.

Results and Discussion

Bead Morphology

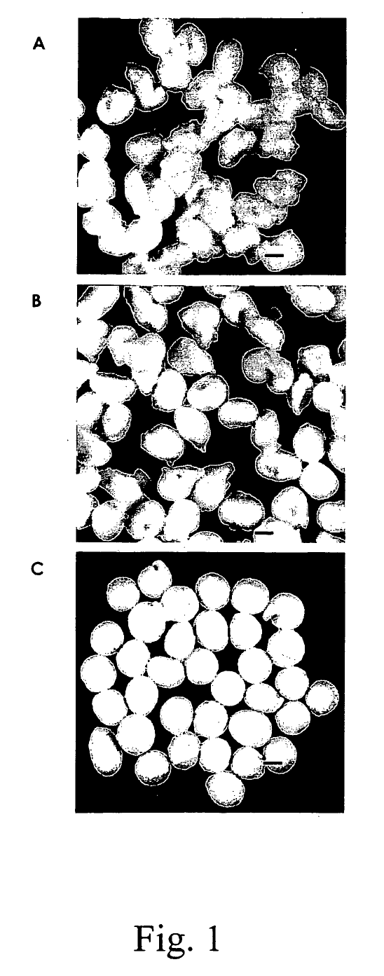

FIGS. 1a to 1c show macrophotographs of whey protein beads prepared with different calcium chloride (CaCl₂) concentrations: 10% (FIG. 1a), 15% (FIG. 1b), and 20% (w/w) (FIG. 1c). The result shows that the CaCl₂ concentration used in the extrusion step has an influence on both the size and appearance of the beads. Indeed, when the CaCl₂ concentration increases from 10 to 20%, the size of the beads decreases from 2.1 to 1.8 mm. Moreover, the shape of the beads becomes more regular and spherical with higher concentrations. At 10% (w/w) concentration, the beads have an irregular shape and aggregate together, while at 15% (w/w) concentration, beads are more round. Conversely, at 20% (w/w) concentration of CaCl₂, the beads are regular and spherical in shape and are characterized by a smooth surface. The increase in sphericity with higher CaCl₂ concentrations is interesting since this characteristic is expected in controlled delivery because it allows a constant release. The higher sphericity with the elevated CaCl₂ concentration may be due to an increase in the kinetic mechanism of gelation with calcium chloride concentration. Indeed, it has recently been shown that this parameter is likely to be major determinant in the aggregation process. Ca²⁺ acts as a bridge between proteins molecules and favors intermolecular interactions resulting in the aggregation process. Moreover, Ca²⁺ binding to unfolded protein molecules causes an increase in the reactive sulfhydryl group content thereby participating more easily in the aggregation process. Therefore, it is likely that the increase in CaCl₂ concentration increases protein-protein interactions and results in further aggregation of the protein to form a gel network.

Internal Microstructure Analyses of Beads by Transmission Electron Microscopy (TEM)

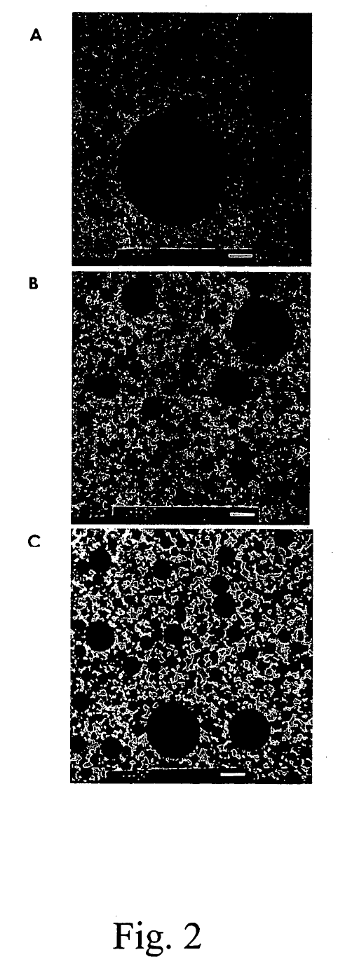

FIGS. 2a to 2c displays microstructures of selected beads prepared with various CaCl₂ concentrations: 10% (FIG. 2a), 15% (FIG. 2b), and 20% (w/w) (FIG. 2c). Each image shows a uniform (homogeneous) oil globules distribution in a gel protein network. The micrographs show that increasing CaCl₂ concentration from 10% (w/w) to 20% (w/w) resulted in smaller fat globules and in a more homogeneous network. This suggests that increasing CaCl₂ concentration prevents coalescence of oil droplets in the protein network. It is known that coalescence is a phenomenon that results from the fusion of individual droplet emulsion into bigger droplets and leads to an increase in average sphere size. During the emulsification step of bead formation, thermal pre-denatured proteins, acting as an emulsifier, rapidly adsorb to the surface of the oil droplets. The large negative change in free energy associated with protein adsorption creates a stabilizing layer that protects the fine droplets against coalescence and provides physical stability to the emulsion. In the second step, addition of Ca²⁺ reduces the electrostatic stabilization of the emulsion, which could favor the coalescence of some droplets. Increasing Ca²⁺enhances the gelation kinetic. Thus, the droplets are rapidly trapped and stabilized by the protein network. Attractive electrostatic interactions between adsorbed proteins on adjacent droplets and Ca²⁺ are reinforced by increasing Ca²⁺ levels. Calcium acts as a bridge between adjacent emulsion droplets, and favors their aggregation without disruption of the protective stabilizing protein layer at the interface thereby, preventing their coalescence.

Physicochemical and Mechanical Bead Characterization

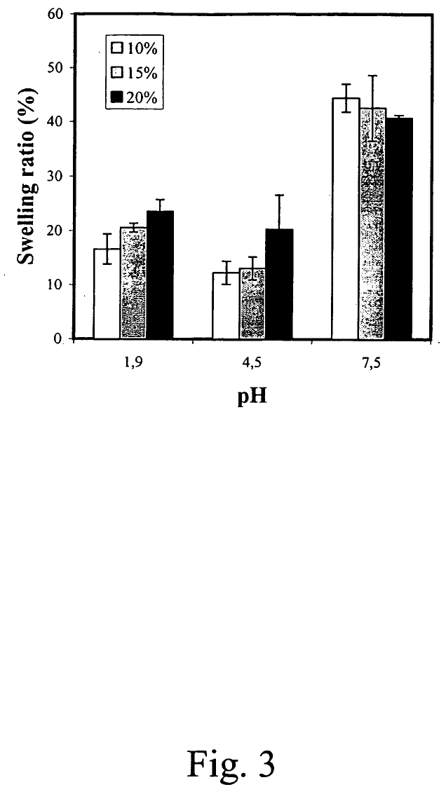

Swelling ratio: FIG. 3 displays the equilibrium-swelling ratio of the beads as a function of CaCl₂ concentration as well as the pH of the swelling medium. The statistical analysis shows that the effect of pH levels on the bead-swelling ratio is influenced by the CaCl₂ concentration (p<0.05). The figure reveals that the pH of the medium has a striking effect on the swelling of the beads. It is at a minimum at pH 4.5, near the pl (5.2) of the whey protein, and increases with changes in pH values (increased—intestinal pH (7.5)—or decreased—gastric pH (1.9)). These results suggest that bead swelling is mainly governed by the net charge of the protein molecules. At pl, the net charge of the whey protein molecule is at a minimum, which translates into low electrostatic repulsions between chains and results in low swelling ratio. The protein-protein interactions are favored by protein-solvent interactions. However, as the pH differs from pl, the net charge of the whey protein molecule increases (positive below pl, negative above pl), leading to high electrostatic repulsive forces and an increase in the swelling ratio. The beads are highly swollen at intestinal pH (7.5). This high equilibrium-swelling ratio can be attributed to the electrostatic repulsive force originating from the negative charge of the ionized carboxyl groups, suggesting that these groups are mainly involved in the pH-sensitive swelling property. At the gastric pH (1.90), the beads are less swollen. This suggests that the low repulsion electrostatic interactions, between positive charges, caused by the protonation of the amine groups on the protein chain, resulted in a low network swelling, but their contribution cannot be ruled out of the pH-sensitive swelling mechanism. It can therefore be concluded that the ionizable and/or ionized groups are the major factors that govern the pH-sensitive swelling mechanism of the beads. Although CaCl₂ does not have a significant effect on the swelling ratio, we can note a trend of a higher swelling at 20% CaCl₂. This may be due to a more homogeneous protein network at this concentration, as seen by the internal bead structure, which improves the water-trapping capacity of the gel.

Rupture Strength

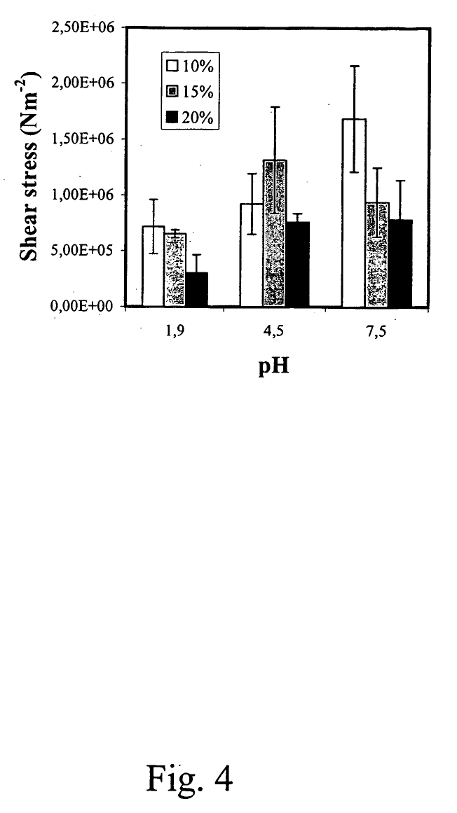

FIG. 4 shows the results of the measurements of the stress at fracture (Nm⁻²) as a function of CaCl₂ concentration and pH. The statistical analysis shows that the effect of environment pH on shear stress at bead failure depended on the CaCl₂ concentration (p<0.05). The figure shows that higher pH values increase the shear stress of the beads. The shear stress is smaller at pH 1.9 and is relatively constant at pH 4.5 and pH 7.5. Consequently, the resistance at bead failure is higher at both these pH values (4.5, 7.5) compared to pH 1.9. It is interesting to note that at pH 7.5 and 4.5, the beads exhibit similar rupture strengths, despite their different swelling properties. This unexpected result could be explained by interactions in the protein network. As seen before, at pH 4.5, near the isoelectric point of β-lactoglobulin, the protein-protein interactions (aggregates) are favored leading to a high shear stress. The rigid structure of the beads in these conditions increases their hardness. At pH 7.5, repulsive electrostatic interactions, between negative charges, prevented the formation of protein-protein interactions and favored the swelling of the bead internal network. Thus, the resulting elasticity improves the fracture strength of the beads, which adopt a rubber-like behavior. At pH 1.9, the low repulsion electrostatic interactions, between positive charges, caused a low network swelling and allowed a weak shear stress.

The fracture stress is also affected by calcium concentration. Higher calcium concentrations result in lower rupture strength of the beads. The authors showed that increasing CaCl₂ concentration at low protein concentration (<10%), lowered Ca²⁺-induced cold gel strength. It is likely that the change in CaCl₂ concentration affects the association/dissociation equilibrium of Ca²⁺ binding to the proteins. Moreover, at low CaCl₂ concentrations, it can be suggested that, the heterogeneity of the network, due to the presence of big fat globules, leads to the development of network areas where protein-protein interactions are reinforced as well as other highly elastic areas that result in higher rupture strength.

Fracture Strain

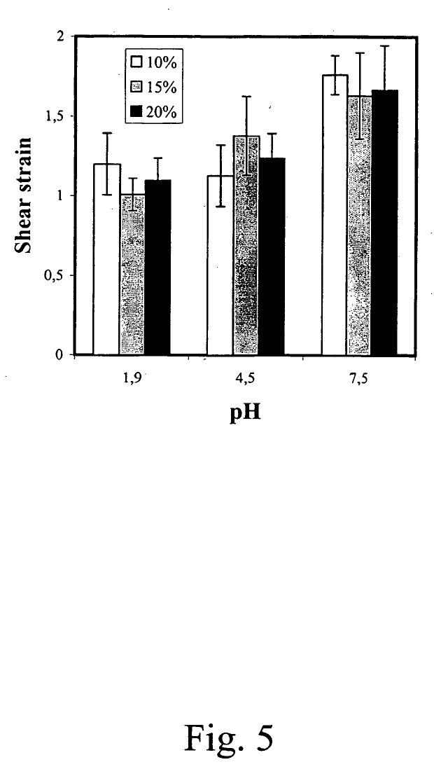

FIG. 5 presents the results of the measurements of shear strain as a function of CaCl₂ concentration and pH. Statistical analysis revealed no significant interaction (p>0.05), between pH and CaCl₂ concentration. The figure shows that bead deformability is relatively constant at pH 1.9 and 4.5, and increases at pH 7.5. As expected, the high swelling ratio obtained at pH 7.5 allows a greater deformability compared to other pH values. As seen in the figure, the concentration of CaCl₂ does not significantly affect shear strain at failure even though lower values were observed at 20% CaCl₂ concentration.

Stress Relaxation

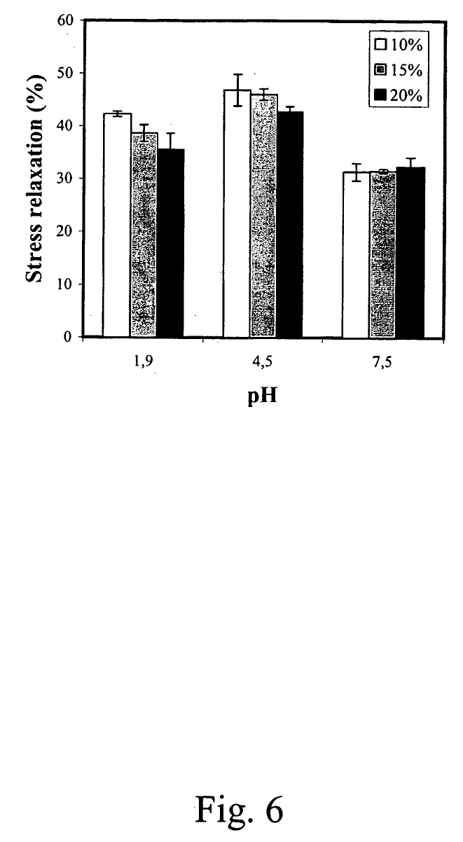

FIG. 6 shows the results of the measurements of stress relaxation as a function of CaCl₂ concentration and pH. The effect of environment pH on stress relaxation of the beads depended on the CaCl₂ concentration (p<0.001). The beads stress relaxation increases with pH, up to a maximal value obtained with pH 4.5. Then the stress relaxation considerably decreased at pH 7.5. This means that beads exhibit a higher elasticity at pH 7.5 and a lower one at pH 4.5. These results concur with those previously obtained for swelling properties. This result might be explained by the effect of the net charge of the protein molecules that favors, depending on its value, either protein-protein or protein-solvent interactions. As seen before, the type of interaction in the protein networks influences the swelling properties of beads and, therefore, their elasticity, which is favored by the swelling of protein network at pH 7.5.

Increasing CaCl₂ concentration decreases stress relaxation. Consequently, beads have a better elasticity at 20% CaCl₂ concentration, possibly because of the internal bead structure, and this confirms the previous explanation. Globule distribution homogeneity in the protein network conduces to improved flexibility and favors bead elasticity.

Enzymatic Degradation

Beads prepared with CaCl₂ 20% (w/w) were degraded using a method that consisted in a two-step proteolysis performed at 37° C., and included a pepsin predigestion at pH 1.9, followed by hydrolysis with pancreatic enzymes at close to neutral pH.

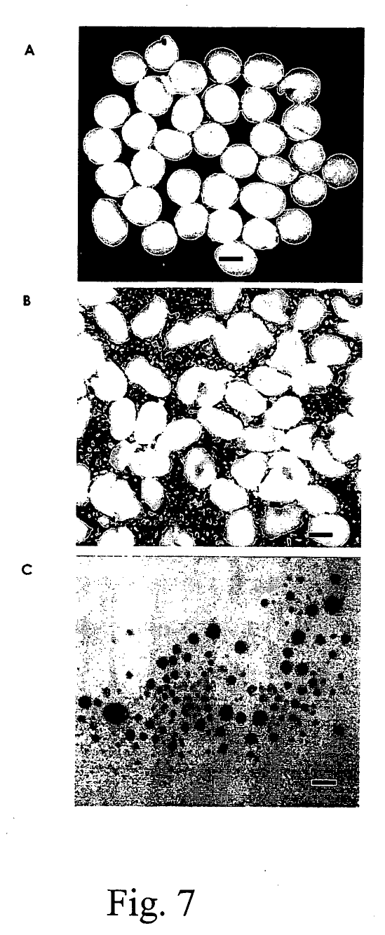

FIGS. 7a to 7c show macrophotographs of beads during in vitro digestion: intact bead (FIG. 7a), after gastric incubation (Fig. b), and after pancreatic incubation (Fig. c). This figures reveal that the beads exhibit a resistance to pepsin hydrolytic action, but are totally degraded in pancreatic media. Indeed, macroscopic bead examination, before and after gastric incubation, shows a very slight degradation suggesting that the beads are gastro-resistant. As for enzymatic specificity, pepsin is known to preferentially attack peptide bonds involving hydrophobic aromatic amino-acids. In its native structure, the major protein of whey, β-lactoglobulin (β-Ig), it is resistant to pepsin since its hydrophobic amino acids are located in the internal core of its calyx-like structure. In the initial step of bead formation, the protein molecules are heated above their thermal denaturation temperature leading to a disruption of both their tertiary and the H-bonded secondary structures. The primary importance of the denaturation process is to expose functional groups, such as CO and NH of peptide bonds, side-chain amide groups, and hydrophobic amino acids. The thermal denaturation of whey proteins was therefore expected to cause a significant increase in the susceptibility of proteins to proteolysis degradation, particularly as far as peptic digestion is concerned. However, in the emulsification step of bead formation, the hydrophobic amino acids, adsorb at the surface of the oil droplets, that are trapped in the protein network by adding Ca²⁺. The hydrophobic amino acids are thus masked, which prevents the action of pepsin.

As for degradation by pancreatin, beads were completely destroyed within. 6 hours. After this incubation time, only fat globules remained in the solution. This degradation by pancreatin would then be attributed to the combined effect of the proteases, mainly trypsin, chymotrypsin, and elastase, which catalyze the hydrolysis of the peptide (amine) bonds, but with different specificities. The action of trypsin is known to be restricted to the peptide links that involve the carboxylic groups of lysine and arginine, chymotrypsin is specific to bulky hydrophobic residues preceding the scissile peptide bond, and elastase is specific to small neutral residues.

It can therefore be concluded that bead degradation is mainly enteric and that these beads can be useful as matrix to protect fat-soluble bioactive molecules sensitive to stomach pH.

This work has allowed the development of a new encapsulation method that exploits protein emulsification and gelation properties. The emulsification/cold gelation procedure outlined in the present demonstration illustrates an innovative technique for producing protein beads. Their physicochemical, mechanical, and degradation properties may be modulated. First, Ca²⁺ modulated the spherical shape of the beads as well as their characteristics: at a high calcium chloride concentration, beads have a lower shear stress and a better elasticity. The gel aggregation is affected by the conditions of the gelation process. Secondly, bead hydration is dependent on the pH medium and involves an improvement of elasticity. At high water content, resistance at fracture could be elevated. Bead protein chains reorganize their interactions according to environmental conditions. Lastly, bead degradation is mostly enteric. It thus, seems likely that beads are not susceptible to enzymatic attack during a rapid transit in the stomach; the action is prevented by the bead structure.

The results of this research demonstrate that beads at a 20% (w/w) CaCl₂ concentration presented an excellent capacity to encapsulate bioactive molecules that are hydrophobic and sensitive to stomach pH. These spherical and elastic beads are composed of a homogeneous distribution of globules in a protein network. These beads therefore appear as promising matrices with applications in various fields such as food, nutraceutics, pharmaceutics, and cosmetics.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A process for making protein particles for delivery of a bioactive molecule or living system comprising the step of:
a) providing a solution of protein;
b) heating said solution of step a) to a temperature between about 20° C. to 150° C. for a period of time from at least 2 minutes to 10 hours to allow denaturation of said protein;
c) adding a hydrophobic phase to said heated solution of step b) in a ratio of between about 5 to 60 percents (vol/vol) to form a mixed solution, and agitating said mixed solution so that an emulsion is formed;
d) homogenizing said emulsion of step c); and
e) contacting said homogenized emulsion of step d) with a salt solution to induce formation of protein particles.

2. The process as claimed in claim 1, wherein said protein of step a) is selected from the group consisting of synthetic peptide, milk protein, whey protein, vegetable protein, bran protein, animal protein, and globular peptide or protein.

3. The process as claimed in claim 1, wherein said heated solution of step b) is cooled down before addition of said hydrophobic phase of step c).

4. The process as claimed in claim 1, wherein said homogenization is dynamic high pressure, or mechanical homogenization.

5. The process as claimed in claim 1, wherein said step c) further comprises addition of at least one of a physiological agent, a bioactive molecule or a living system.

6. The process as claimed in claim 5, wherein said living system is selected from the group consisting of bacteria, virus, mould, yeast, semen, pollen, grain, and microorganism.

7. The process as claimed in claim 1, wherein said hydrophobic phase of step c) is selected from the group consisting of oil, physiologically acceptable carrier, adjuvant, emulsifier, diluent or excipient.

8. The process as claimed in claim 7, wherein said oil is selected from the group consisting of animal, mineral, and vegetable oil.

9. The process as claimed in claim 1, wherein said bioactive molecule is selected from the group consisting of nutraceutical, immunological, enzymatic, cosmetic, cosmeceutical, and therapeutical agents.

10. The process as claimed in claim 1, wherein said delivery is cutaneous application or oral administration.

11. The process as claimed in claim 10, wherein said subject is a human or an animal.

12. The process as claimed in claim 1, wherein said delivery is addition to a composition selected from the group consisting of an organic mixture, a fertilizer, a manure, a earth, a ground, or a land.

13. The process as claimed in claim 1, wherein said salt of step e) is a soluble salt selected from the group consisting of divalent cations, calcium chloride, calcium phosphate, sodium phosphate, sodium carbonate, potassium carbonate, calcium sulfate, carboxylic acid, salts, barium, magnesium, calcium, iron, and derivatives thereof.

14. A protein particle for delivery of a bioactive agent or system to a subject or a composition, said particle obtained by heating a protein solution to a temperature between about 20° C. to 150° C. for a period of time from at least 2 minutes to 10 hours to allow denaturation of said protein; adding a hydrophobic phase to said heated solution in a ratio of between about 5 to 60 percents (vol/vol) to form a mixed solution, and agitating said mixed solution so that an emulsion is formed; homogenizing said emulsion; and contacting said homogenized emulsion with a salt solution to induce formation of protein particles.

15. The protein particle as claimed in claim 14, wherein said protein of step a) is selected from the group consisting of synthetic peptide, milk protein, whey protein, vegetable protein, bran protein, animal protein, and globular peptide or protein.

16. The protein particle as claimed in claim 14, wherein said heated solution is cooled down before addition of said hydrophobic phase.

17. The protein particle as claimed in claim 14, wherein said homogenization is dynamic high pressure, or mechanical homogenization.

18. The protein particle as claimed in claim 14, wherein said addition of hydrophobic phase is combined to addition of at least one of a physiological agent, a bioactive molecule or a living system.

19. The protein particle as claimed in claim 18, wherein said living system is selected from the group consisting of bacteria, virus, mould, yeast, semen, pollen, grain, and microorganism.

20. The protein particle as claimed in claim 14, wherein said hydrophobic phase is selected from the group consisting of oil, physiologically acceptable carrier, adjuvant, emulsifier, diluent or excipient.

21. The protein particle as claimed in claim 20, wherein said oil is selected from the group consisting of animal, mineral, and vegetable oil.

22. The protein particle as claimed in claim 14, wherein said bioactive molecule is selected from the group consisting of nutraceutical, immunological, enzymatic, cosmetic, cosmeceutical, and therapeutical agents.

23. The protein particle as claimed in claim 14, wherein said delivery is cutaneous application or oral administration.

24. The protein particle as claimed in claim 14, wherein said subject is a human or an animal.

25. The protein particle as claimed in claim 14, wherein said delivery is addition to a composition selected from the group consisting of an organic mixture, a fertilizer, a manure, a earth, a ground, or a land.

26. The protein particle as claimed in claim 14, wherein said salt of is a soluble salt selected from the group consisting of divalent cations, calcium chloride, calcium phosphate, sodium phosphate, sodium carbonate, potassium carbonate, calcium sulfate, carboxylic acid, salts, barium, magnesium, calcium, iron, and derivatives thereof.

27. A method for delivery of a bioactive molecule or a living system to a subject or a composition comprising administrating to said subject or composition a protein particle as defined in claim 14 containing at least one bioactive molecule or living system.

28. The method as claimed in claim 27, wherein said administration is cutaneous application, oral administration.

29. The method as claimed in claim 27, wherein said composition is selected from the group consisting of a fertilizer, earth, land or ground.

30. The method as claimed in claim 27, wherein said subject is a human or an animal.

31. Use of a protein particle as defined in claim 14 in the preparation of a delivery system for delivery a bioactive molecule or a living system to an animal, a human or a composition selected from the group consisting of a fertilizer, earth, land or ground.
